(19) United States
(12) Patent Application Publication
(10) Pub. No.: US 2005/0215620 A1
(43) Pub. Date: Sep. 29, 2005

(54) Method of treating amyloid beta precursor disorders

(75) Inventor: Lawrence Friedhoff, Hackensack, NJ (US)

Correspondence Address: DAVIDSON, DAVIDSON & KAPPEL, LLC, 485 SEVENTH AVENUE, 14TH FLOOR, NEW YORK, NY, 10018, US

(73) Assignee: Andrx Labs, LLC, Davie, FL (US)

(21) Appl. No.: 11/072,350

(22) Filed: Mar. 4, 2005

Related U.S. Application Data
(63) Continuation of application No. 09/704,554, filed on Nov. 3, 2000, now abandoned.
(60) Provisional application No. 60/163,608, filed on Nov. 4, 1999.
(60) Provisional application No. 60/219,435, filed on Jul. 20, 2000.
(60) Provisional application No. 60/223,987, filed on Aug. 9, 2000.

(51) Int. Cl. A61K 31/401; A61K 31/366; A61K 31/225

(52) U.S. Cl. 514/423; 514/460; 514/548

(57) ABSTRACT

Methods for the treatment and prevention of APP processing disorders such as Alzheimer's disease and Down's Syndrome which are based on the administration of an effective amount of a HMG-CoA reductase inhibitor to a mammal are disclosed. Additionally, methods for the treatment and prevention of APP processing disorders such as Alzheimer's disease and Down's Syndrome which are based on the reduction of cellular cholesterol in a mammal are disclosed. These methods reduce the amount of Aβ peptides or decrease the formation of Aβ peptides or increase the clearance of Aβ peptides in a mammal suffering from Alzheimer's disease and Down's Syndrome.

### RELATED APPLICATION DATA

This application claims the benefit of U.S. Provisional Patent Application No. 5 60/163,608, filed Nov. 4, 1999, U.S. Provisional Patent Application No. 60/219,435, filed Jul. 22, 2000, and U.S. Provisional Patent Application No. 60/223,987, filed Aug. 9, 2000, naming Drs. Lawrence Friedhoff and Joseph Buxbaum as co-inventors. The aforementioned applications are herein incorporated by reference.

### TECHNICAL FIELD

The present invention relates to a method of treating amyloid β precursor protein (APP) disorders such as Alzheimer's disease and Down's Syndrome.

### BACKGROUND OF THE INVENTION

The cause of Alzheimer's disease is not known. The disease is characterized by the accumulation of β -amyloid peptides (Aβ peptides), as abnormal protein precipitates, in the brain. It is generally believed that these proteins kill brain cells which causes a loss of mental function.

As illustrated in FIG. 1, immature amyloid β precursor protein (APPᵢ) under-goes glycosylation to become mature amyloid β precursor protein (APPₘ). Then APPₘ is either (1) cleaved by the protease α-secretase to produce a secreted form of APP (APPₛ) which is not amyloidogenic, or (2) cleaved by β-secretase and γ-secretase to produce the abnormal protein, Aβ (Aβ peptide), which can then precipitate.

Many advances have been made in the treatment of Alzheimer's disease. The cholinesterase inhibitors such as tacrine, donepezil and rivastigmine improve symptoms slightly. However, the slight improvement in attention and alertness is most likely due to increased brain acetylcholine levels. Unfortunately, however, the cholinesterase inhibitors do not prevent cognitive decline, which is inevitably fatal even with optimal cholinesterase inhibitor treatment.

Several strategies for treating Alzheimer's disease have been proposed and include decreasing or preventing the release of Aβ peptide by either increasing α-secretase or decreasing the β- or γ-secretase activity or production. Other strategies include decreasing Aβ peptide aggregation, increasing Aβ peptide clearance, reducing Aβ peptide production or decreasing the cellular effects of Aβ peptide aggregation and deposition. See Sabbagh, M N. et al., (1997) Alzheimer's Disease Rev. 3:1-19. See also U.S. Pat. No. 6,080,778. In light of the foregoing, there is a need for a more effective treatment of mammals suffering from APP processing disorders such as Alzheimer's disease and Down's Syndrome.

### SUMMARY OF THE INVENTION

Generally, the invention relates to a method for treating a mammal having an APP processing disorder comprising administering to the mammal a composition comprising a therapeutically effective amount of at least one HMG-CoA reductase inhibitor. APP processing disorders include Alzheimer's disease and Down's Syndrome.

In a preferred embodiment, the invention relates to a method of treating a mammal having Alzheimer's disease and/or Down's Syndrome by administering to the mammal a therapeutically effective amount of at least one HMG-CoA reductase inhibitor. In this embodiment, the method may also comprises determining whether the mammal exhibits at least one objective symptom of Alzheimer's disease or Down's Syndrome.

In another embodiment of the present invention, the composition comprising at least one HMG-CoA reductase inhibitor may further comprise a pharmaceutically acceptable excipient. The composition is preferably in the form of a controlled release formulation.

In a preferred embodiment of the present invention, the HMG-CoA reductase inhibitor is selected from the group consisting of mevastatin, pravastatin, simvastatin, atorvastatin, lovastatin, rivastatin and fluvastatin, and pharmaceutically effective salts, isomers and the active metabolite forms thereof, or a combination thereof. In a more preferred embodiment, the HMG-CoA reductase inhibitor is lovastatin or lovastatin acid.

In another preferred embodiment, about 0.2 mg to about 10 mg of the HMG-CoA reductase inhibitor per Kg of the mammal's body weight per day is administered. The daily amount administered to the mammal may be administered in more than one fraction.

In another preferred embodiment, an oral dose of about 5 mg to about 400 mg of lovastatin per day is administered to a human having an APP processing disorder. In a more preferred embodiment the oral dose is about 10 mg to about 350 mg per day. More preferably, the oral dose is about 10 mg to about 300 mg per day. Even more preferably, the oral dose is about 10 mg to about 250 mg per day.

In another preferred embodiment, any suitable dose of an HMG-CoA reductase inhibitor is administered to a mammal having an APP processing disorder. More preferably, the suitable dose is one that is therapeutically effective and results in the average blood plasma concentration of the HMG-CoA reductase inhibitor or its active metabolite at steady-state being below about 50 micromolar. More preferably, the blood plasma concentration of the HMG-CoA reductase inhibitor or its active metabolite at steady-state is below about 30 micromolar. Even more preferably, the blood plasma concentration of the HMG-CoA reductase inhibitor or its active metabolite at steady-state is below about 20 micromolar. In an even more preferred embodiment, the blood plasma concentration of the HMG-CoA reductase inhibitor or its active metabolite at steady-state is below about 10 micromolar. Even more preferably, the blood plasma concentration of the HMG-CoA reductase inhibitor or its active metabolite at steady-state is below about 5 micromolar. Even more preferably, the blood plasma concentration of the HMG-CoA reductase inhibitor or its active metabolite at steady-state is below about 1 micromolar. Most preferably, the blood plasma concentration of the HMG-CoA reductase inhibitor or its active metabolite at steady-state is about 0.5 micromolar.

In another embodiment, the invention relates to a method for treating a mammal having an APP processing disorder which comprises lowering the amount of Aβ peptides in the brain, cerebral spinal fluid, or plasma of the mammal by administering to the mammal a composition comprising a therapeutically effective amount of at least one HMG-CoA reductase inhibitor. Lowering the amount of Aβ peptides in the brain may comprise affecting APPₘprocessing. In a preferred embodiment, the amount of Aβ peptides is lowered in the brain of the mammal.

In another embodiment, the invention relates to a method for treating a mammal having an APP processing disorder which comprises increasing the clearance of Aβ peptides in the brain, cerebral spinal fluid, or plasma of the mammal by administering to the mammal a composition comprising a therapeutically effective amount of at least one HMG-CoA reductase inhibitor. In a preferred embodiment, the clearance of Aβ peptides in the brain of the mammal is increased.

In another embodiment, the invention relates to a method for treating a mammal having an APP processing disorder comprising preventing or reducing Aβ peptide aggregation or plaque formation in the brain of the mammal by administering to the mammal a composition comprising a therapeutically effective amount of at least one HMG-CoA reductase inhibitor.

In another embodiment, the invention relates to a method for the treatment of a mammal exhibiting the objective symptoms of Alzheimer's disease by decreasing the formation of Aβ peptides, increasing the clearance of Aβ peptides, regulating the processing of APP, or reducing plaque maturation in the mammal by administering to the mammal a composition comprising a therapeutically effective amount of at least one HMG-CoA reductase inhibitor.

In another embodiment, the invention relates to a method for treating a mammal having an APP processing disorder comprising lowering the amount cellular cholesterol levels in the mammal. In a preferred embodiment, the amount of cellular cholesterol levels are decreased by the administration of at least one HMG-CoA reductase inhibitor.

Generally an immediate release or a controlled release dosage form may be utilized in the practice of the invention. The immediate release dosage formulation may comprise an effective amount of a HMG-CoA reductase inhibitor and a suitable pharmaceutical diluent. The controlled release dosage formulation may comprise:
a compressed tablet core which contains an alkyl ester of a hydroxy substituted naphthalene derivative, a pharmaceutically acceptable, water swellable polymer and an osmotic agent; and
an outer coating layer which covers the osmotic core and comprises a pH sensitive coating agent and a water insoluble polymer.

An optional sealing coat may be applied to the compressed tablet core and an optional coating layer comprising an enteric coating agent may be applied under the outer coating layer as an inner coating or as an overcoat over the outer coating layer. The tablet core may be compressed using a smooth faced tablet die. The preferred alkyl ester of a hydroxy substituted naphthalene compound is lovastatin. Plasma levels of about 0.5 micromoles of the HMG-CoA reductase inhibitor are preferably maintained by the use of a controlled release formulation of the HMG-CoA reductase inhibitor.

### BRIEF DESCRIPTION OF THE DRAWINGS

FIG. 1 is a schematic which illustrates APP processing.

FIGS. 2a and 2b illustrate the effects of lovastatin acid on Aβ peptides in Human Neuroglioma (H4) cells. FIG. 2a is a photograph of two gel wells wherein the negative well and the positive well correspond to the bar graphs of FIG. 2b representing 0 and 0.5 μM of lovastatin acid, respectively. Data represent the mean±the standard error of the mean (SEM) of one experiment performed in quadruplicate.

FIGS. 3a and 3b illustrate the effects of lovastatin acid on Aβ peptides in Madin-Darby Canine Kidney (MDCK) cells. FIG. 3a is a photograph of two gel wells wherein the negative well and the positive well correspond to the bar graphs of FIG. 3b representing 0 and 0.5 μM of lovastatin acid, respectively. Data represent the mean ±SEM of three experiments performed in quadruplicate.

FIGS. 4a and 4b illustrate the effects of lovastatin acid on Aβ peptides in Chinese Hamster Ovary (CHO) cells. FIG. 4a is a photograph of two gel wells wherein the negative well and the positive well correspond to the bar graphs of FIG. 4b representing 0 and 0.5 μM of lovastatin acid, respectively. Data represent the mean ±SEM of four experiments performed in quadruplicate.

FIG. 5 illustrates the effects of lovastatin acid on APPₛ processing. Data represent the mean ±SEM of an experiment performed in quadruplicate.

FIG. 6 illustrates the effects of lovastatin acid on mature APP processing. Data represent the mean ±SEM of an experiment performed in quadruplicate.

FIG. 7 is a graph showing the steady-state plasma concentrations of lovastatin acid in patients after multiple oral 40 mg doses of Lovastatin XL, a preferred extended release tablet form of lovastatin.

FIG. 8 is a graph showing the change in the mean Aβ peptide concentration in the blood of groups of patients after treatment with various doses of Lovastatin XL.

FIG. 9 is a bar chart showing the change in the mean Aβ peptide concentration in the blood of groups of patients after treatment with various doses of Lovastatin XL.

### DETAILED DESCRIPTION OF THE INVENTION

Recently, the present inventors have discovered that HMG-CoA reductase inhibitors lower the amount of Aβ peptide levels, prevent or reduce Aβ peptide formation, may increase Aβ clearance, and therefore prevent or reduce Aβ peptide aggregation. More particularly, the present inventors have discovered that the administration of HMG-CoA reductase inhibitors lower the amount of Aβ peptide levels, prevent or reduce Aβ peptide formation, may increase Aβ clearance, and therefore prevent or reduce Aβ peptide aggregation, without the need of other cholesterol lowering treatments. Therefore, methods of treating APP processing disorders such as Alzheimer's disease and Down's Syndrome in a mammal comprising the administration of a HMG-CoA reductase inhibitor to the mammal is disclosed herein below.

As used herein, “APPᵢ” means the immature form of amyloid β protein precursor, “APPₘ” means the mature form of amyloid β protein precursor, “APPₛ” means the amyloid β protein precursor which is cleaved by α-secretase and which is the secreted form, “APP” means either APPᵢ, APPₘ, or both.

As used herein, “post-translational” events include the cleavage of APPₘ by β- and γ-secreatases.

As used herein, “other cholesterol lowering treatments” means any treatment other than treatment with a HMG-CoA reductase inhibitor. Other cholesterol lowering treatments include, but are not limited to, treatment with mevalonate, methyl-β-cyclodextrin, and/or cyclodextrin.

As used herein, “active metabolite” is intended to mean a pharmacologically active product produced through metabolism in the body of a specified compound or salt thereof. Active metabolites of a compound may be identified using routine techniques known in the art. See, e.g., Bertolini, G. et al., J Med. Chem., 40, 2011-2016 (1997); Shan, D. et al., J Pharm. Sci., 86 (7), 765-767; Bagshawe K., Drug Dev. Res., 34, 220-230 (1995); Bodor, N., Advances in Drug Res., 13, 224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991).

As used herein, the term “pharmaceutically acceptable salts” refers to salt forms that are pharmacologically acceptable and substantially non-toxic to the subject being administered the composition of the present invention. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary anmmonium hydroxide such as tetramethylammonium hydroxide), those derived from inorganic bases such as alkali or alkaline earth-metal (e.g., sodium, potassium, lithium, calcium, or magnesium) hydroxides, and those derived from organic bases such as amines, benzylamines, piperidines, and pyrrolidines.

Any HMG-CoA reductase inhibitor may be used in the method of the present invention. The term “HMG-CoA reductase inhibitor” refers to any one or more compounds that inhibit the bioconversion of hydroxymethylglutamyl-coenzyme A to mevalonic acid which is catalyzed by the enzyme HMG-CoA reductase. Such inhibition may be determined by standard methods known to those of ordinary skill in the art. Examples of suitable HMG-CoA reductase inhibitors are described and referenced herein, however, other HMG-CoA reductase inhibitors will be known to those of ordinary skill in the art. Therefore, the present invention should not be limited to the specific HMG-CoA reductase inhibitors exemplified herein.

Examples of such HMG-CoA reductase inhibitors which are useful in the method of the present invention for the treatment of Alzheimer's disease include mevastatin which is described in U.S. Pat. No. 3,671,523; lovastatin which is described in U.S. Pat. No. 4,231,938; pravastatin which is described in U.S. Pat. No. 4,346,227; simvastatin which is described in U.S. Pat. No. 4,444,784; atorvastatin which is described in U.S. Pat. No. 4,647,576; rivastatin which is described in European Pat. No. 491226A; and fluvastatin which is described in U.S. Pat. No 4,739,073. All of these patents are incorporated herein by reference. Further, any suitable isomers of the exemplified HMG-CoA reductase inhibitors may be used, including stereoisomers, enantiomers, or mixtures thereof and, thus, their use in pharmaceutical formulations for the treatment of APP disorders are within the scope of the invention.

Lovastatin is a metabolite which is produced by the natural fermentation of a fungus of the Aspergillus genus. The other compounds of this class are derived from natural and synthetic sources using well known procedures and have similar mechanisms of activity.

Any suitable method for administering the HMG-CoA reductase inhibitor may be used. For example, the HMG-CoA reductase inhibitors may be administered orally to a mammal having Alzheimer's disease or Down's Syndrome an effective amount to relieve the symptoms of Alzheimer's disease or Down's Syndrome.

Preferably, the effective amount of the HMG-CoA reductase inhibitor results in the average blood plasma concentrations of the HMG-CoA reductase inhibitor or its active metabolite at steady-state being below about 50 micromolar. More preferably, the blood plasma concentration of the HMG-CoA reductase inhibitor or its active metabolite at steady-state is below about 30 micromolar. Even more preferably, the blood plasma concentration of the HMG-CoA reductase inhibitor or its active metabolite at steady-state is below about 20 micromolar. In an even more preferred embodiment, the blood plasma concentration of the HMG-CoA reductase inhibitor or its active metabolite at steady-state is below about 10 micromolar. Even more preferably, the blood plasma concentration of the HMG-CoA reductase inhibitor or its active metabolite at steady-state is below about 5 micromolar. Even more preferably, the blood plasma concentration of the HMG-CoA reductase inhibitor or its active metabolite at steady-state is below about 1 micromolar. Most preferably, the blood plasma concentration of the HMG-CoA reductase inhibitor or its active metabolite at steady-state is about 0.5 micromolar.

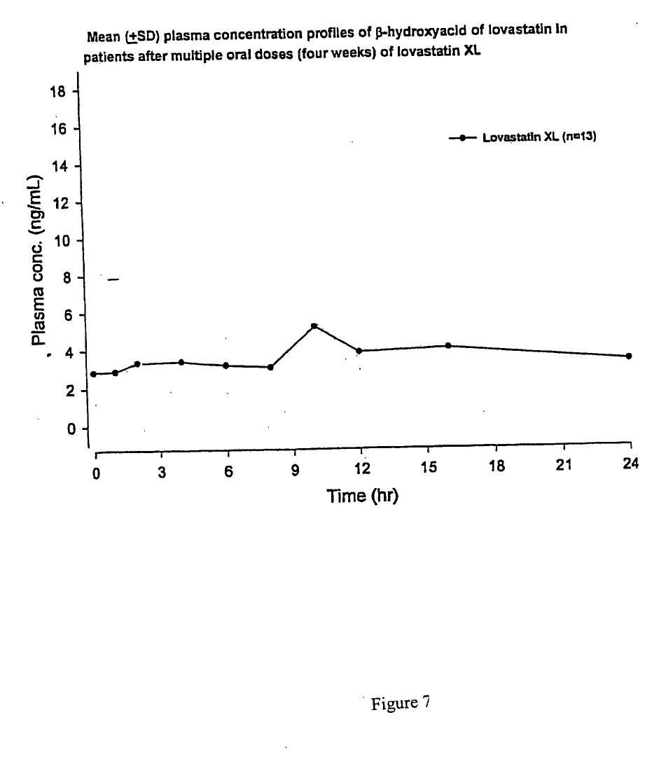

FIG. 7 shows the steady-state plasma concentrations (nanograms/ml) of lovastatin acid in patients after multiple oral 40 mg doses of Lovastatin XL, a preferred extended release tablet form of lovastatin. Accordingly, based on a conversion factor and the known linear pharmacokinetics of lovastatin it can be expected that oral doses of about 233 mg Lovastatin XL (“Lovastatin XL” refers to a lovastatin controlled release formulation as exemplified herein below) given daily to a patient would result in average blood plasma level of the patient being about 0.5 micromolar.

However, the present inventors have surprisingly discovered that human patients given oral doses of only 10 mg/day, 20 mg/day, 40 mg/day and 60 mg/day of Lovastatin XL resulted in a statistically significant decrease in Aβ peptide levels in the blood plasma of those patients. Accordingly, the inventors have unexpectedly found that the HMG-CoA reductase inhibitor may be administered to a human orally at daily doses of about 10 mg to about 60 mg.

Preferably, the HMG-CoA reductase inhibitor is administered to the mammal orally at a daily dose of about 0.2 mg to 10.0 mg per kg of body weight, given in divided doses. The HMG-CoA reductase inhibitors may be administered in any suitable form. For example, the HMG-CoA reductase inhibitor may be administered in the form of tablets, capsules or oral concentrates suitable for mixing the particular compound with food.

The criteria for the diagnosis of Alzheimer's disease is well known and is set forth in the guidelines of the National Institute of Neurological and Communicative Disorders and Alzheimer's Disease and Related Disorders Association (McKhann et al., Neurology 1984; 34: 939-944); and in the American Psychiatric Association, Diagnostic and Statistical Manual of Mental Disorders (Diagnostic and Statistical Manual IV), all of which are incorporated herein by reference. Generally the objective criteria for the diagnosis of Alzheimer's disease include: gradual memory impairment and gradual onset of at least one of the following aphasia, apraxia, agnosia or disturbance of executive functioning.

Treatment may be continued until there is a reduction in the symptoms of Alzheimer's disease and the dosage may be adjusted in response to the mammal's individual response. Generally a positive response will not be seen until therapy has been continued for a minimum period of 90 to 365 days.

More preferably, a controlled release formulation (also herein after referred to as a “controlled release composition”) of the HMG-CoA reductase inhibitor is utilized in order to provide an enhanced effect that cannot be achieved by conventional immediate release dosing. The use of a controlled release form may be specially useful for providing a constant level of the HMG-CoA reductase inhibitor in order to avoid dosage peaks and valleys in those mammals who have meals at irregular times or those who frequently eat snacks between meals.

Controlled release formulations have been described in U.S. Pat. No. 4,615,698 which have been based on an osmotic dosage form which is designed to collapse and cause the faced surfaces to come into a closed contacting arrangement as the drug is delivered through a passageway in the semi-permeable wall of the dosage form. In addition, U.S. Pat. No. 4,503,030 discloses an osmotic dosage form which has a passageway and a semi-permeable membrane consisting of a particular cellulose polymer and a pH sensitive material which could be an enteric coating material. This patent describes the use of 1:1 mixtures of a pH sensitive material and cellulose polymer which are applied at a level of about 7% by weight based on the total weight of the osmotic core tablet and coating material. The aforementioned patents are incorporated herein by reference.

Preferred HMG-CoA Reductase Inhibitor Formulations

A preferred controlled release formulation is disclosed in U.S. Pat. No. 5,916,595, which is incorporated herein by reference. This type of a controlled release dosage form is preferably prepared by combining the HMG-CoA reductase inhibitor with a pharmaceutically acceptable, water swellable polymer and an osmotic agent into a compressed tablet core having an optional first coating for sealing and protection and a second coating comprising a pH sensitive agent water insoluble polymer. More preferably, the HMG-CoA reductase inhibitor is selected from the group consisting of mevastatin, pravastatin, simvastatin, atorvastatin, and lovastatin and the active metabolite forms thereof. Even more preferably, the HMG-CoA reductase inhibitor comprises lovastatin or its active metabolite, lovastatin acid. Mevastatin, pravastatin, simvastatin, atorvastatin, and lovastatin are well known compounds that are described in the prior art including the particular patents which have been cited herein. It is also within the scope of the invention to use mixtures of different alkyl esters of hydroxy substituted naphthalenes.

Specifically, the pharmaceutically acceptable, water swellable polymer and the osmotic agent are combined with the HMG-CoA reductase inhibitor which may be micronized, comicronized or unmicronized or amorphous or crystalline and compressed to form the tablet core. The osmotic agent is any suitable non-toxic pharmaceutically acceptable water soluble compound which will dissolve sufficiently in water and increase the osmotic pressure inside the simple sugars and salts such as sodium chloride, potassium chloride, magnesium sulfate, magnesium chloride, sodium sulfate, lithium sulfate, urea, inositol, sucrose, lactose, glucose, sorbitol, fructose, mannitol, dextrose, magnesium succinate, potassium acid phosphate and the like. The preferred osmotic agent for the tablet core is a simple sugar such as anhydrous lactose in the range of about 0-50% by weight, based on the weight of the compressed, uncoated tablet.

The pharmaceutically acceptable, water swellable polymer may be any pharmaceutically acceptable polymer which swells and expands in the presence of water to slowly release the HMG-CoA reductase inhibitor. These polymers include polyethylene oxide, methylcellulose, hydroxypropylcellulose, hydroxypropylmethylcellulose and the like.

In a preferred embodiment, the water swellable polymer will be polyethylene oxide (obtained from Union Carbide Corporation under the trade name Polyox WSR Coagulant or Polyox WSR N 80). These materials form a viscous gel in water or other solvent system at a sufficient concentration to control the release of the HMG-CoA reductase inhibitor. This will generally require a concentration of the pharmaceutically acceptable water swellable polymer of about 0-50% by weight of the compressed, uncoated tablet.

Any suitable binder may be employed. Preferably, the binder is used in a sufficient amount so that when it is combined with a suitable solvent, mixed with the water soluble osmotic agent and agitated, granules will be formed which may be compressed into a tablet core. Prior to compressing the granules, the conventional solid pharmaceutical diluents such as microcrystalline cellulose, lactose, dextrose and the like may be added to the granule based on the weight of the compressed uncoated tablet. In the present case, the above mentioned osmotic agent, lactose, may function as a binder in the tablet compression step.

In the preparation of the tablets, any suitable solvent may be used to prepare the aforementioned granules. In addition, various other suitable diluents, excipients, lubricants, dyes, pigments, dispersants, emulsifiers, and the like may be used to optimize the HMG-CoA reductase inhibitor formulation.

Additionally, any suitable surfactant may be used. The surfactant may be any ionic or non-ionic water soluble surfactant which is preferably employed in the range of about 0-50% by weight and more preferably employed in the range of about 1-5% by weight. The preferred surfactant for the present formulation is sodium lauryl sulfate but other surfactants such as polysorbate 20, 60, or 80; polyoxl 40 stearate and the like may be used.

Furthermore, a tabletizing formulation may also include any suitable lubricant. Ideally, the lubricant will be in the range of from about 0.5 to about 2.5% by weight of the compressed, uncoated tablet.

After the above described tablet core is formed, it is preferably coated with: 1) an optional protective first coating on the tablet core and/or an optional pH sensitive coating; and 2) an outer coating comprising a pH sensitive agent and a water insoluble polymer.

Specifically, a protective first coating may be used at a level in the range of about 0-10% by weight which may be applied from a coating system such as OPADRY CLEAR™ sold by Colorcon Corporation. In an especially preferred embodiment, the OPADRY CLEAR™ will be about 2.83% by weight and will be combined with an osmotic agent in the range of about 0-10% by weight. While the osmotic agent may be any suitable salt, low molecular weight molecule or water soluble polymer, the preferred osmotic agent is sodium chloride. Preferably, the osmotic agent is added to the coating system when the coating system is being dispersed into purified water. The coating system which contains the osmotic agent may then be sprayed onto the tablets to form a protective coating layer.

An optional inner or over coat over the outer coat may also be applied which comprises a pH sensitive polymer which functions as an enteric polymer in that it does not begin to dissolve until pH conditions in excess of the stomach region are encountered. Generally, the pH sensitive materials do not dissolve and begin to release the active drug until the pH is about 3.0, and preferably above about 5.5. Materials such as Eudragit L (copolymer of poly(methacrylic acid, methylmethacrylate), 1:1 ratio; MW (No. Av. 135,000 -USP Type A) or Eudragit S (copolymer of poly(methacrylic acid, methylmethacrylate, 1:2 ratio MW (No. Av. 135,000 -USP Type B) may be used. Hydroxypropyl methyl cellulose phthalate and the like may be used in the range of about 0-30% by weight and preferably about 2 to about 4% by weight of the combined weight of the compressed, uncoated tablet and the inner coating of the pH sensitive polymer.

Preferably, the outer coating comprises a pH sensitive polymer which functions as an enteric polymer in that it does not begin to dissolve until pH conditions in excess of the pH of the stomach region are encountered and a water insoluble polymer which provide controlled release properties to the coating formulation. The pH sensitive polymer is preferably the same type of material that is described above as the optional inner coating layer. The water insoluble polymer may be a cellulosic polymer such as ethylcellulose, cellulose acrylate, cellulose mono-, di- or triacetate. The pH sensitive polymer and the insoluble cellulosic polymer are used at a weight ratio of about 0.1:1 to about 0.75:1, preferably about 0.25:1 to about 0.5:1 of pH sensitive polymer to water insoluble cellulosic polymer. A combined coating weight of about 0.5-5% by weight and preferably about 1-4% by weight and especially preferred is about 1-3% by weight of the gained weight based on the weight of the coated tablet core. Cellulose acetate is the preferred water insoluble polymer and the outer coating is preferably applied as a suspension in acetone.

Furthermore, any suitable plasticizer or combination of plasticizers may be added to the inner, outer or over coating to provide elasticity and shape to the coating. While the plasticizer or combination of plasticizers may be any water soluble or water insoluble formulation in the range of about 0-10% by weight and preferably about 0.5-5% by weight of the outer coating composition. Acetyltributyl citrate is the preferred plasticizer but material such as acetyl triethyl citrate, dibutyl phthalate, triacetin, diethyl phthalate, polyethylene glycol, propylene glycol and the like may be utilized.

Any suitable antioxidant such as butylated hydroxyanisole (BHA) or butylated hydroxytoluene (BHT) may be added to the tablet core as a stabilizer at a level of about 0.001-0.01% by weight of the tablet core.

Any suitable channeling agent may be mixed with the aforementioned components of the outer coating. A channeling agent may be employed to increase the porosity of the film coating in order to increase the amount of the fluids that penetrate the tablet core and increase the rate of hydration. This allows the release of the HMG-CoA reductase inhibitor after the outer film coat ruptures. Generally, channeling agents may be any salts, surfactants, or short-chain water soluble polymers in a water channel forming effective amount, i.e., about 1-5% by weight, based on the total weight of the core and all coating components. The channeling agents include any pharmaceutically acceptable water soluble salt, surfactant, or short-chain water soluble polymer such as sodium chloride, potassium chloride, sucrose, polysorbate-80, hydroxypropyl cellulose, hydroxyethyl cellulose and the like.

Also, the inner or over coating may be supplied with an anti-sticking agent such as talc to overcome any tablet to tablet stickiness during the coating process. The amount of anti-sticking agent supplied is preferably in an amount which prevents sticking, more preferably in the range of about 0-6% by weight based on the weight of the tablets and the coating materials on a dry weight basis.

The tablets may be made by any suitable method, for example, in a smooth faced tablet die. Thereafter the tablet is preferably provided with the outer coating, which because of surface tension, will result in a thinner coating layer over the corners of the tablet which will provide an area in the outer coating which will form a channel to allow intestinal fluid to reach the core of the tablet.

A preferred control release tablet useful in the practice of the present invention will have the following general formula as set forth in Table 1:

TABLE 1

| Tablet Core: | | |
|---|---|---|
| Alkyl ester of a substitute naphthalene | 3-20 | wt % |
| Water Swellable Polymer | 10-40 | wt % |
| Antioxidant | 0.001-0.01 | wt % |
| Osmotic Agents | 20-80 | wt % |
| Surfactant | 0-5 | wt % |
| Lubricant | 0-5 | wt % |
| Coatings: | | |
| Seal Coating | 0-10 | wt % |
| Osmotic Agents | 0-10 | wt % |
| Inner Coating: | | |
| Enteric Polymer | 0-30 | wt % |
| Anti-sticking Agent | 0-6 | wt % |
| Plasticizer | 0-6 | wt % |
| Channeling Agents | 0-6 | wt % |
| Outer Coating: | | |
| Blend of Enteric Polymer and Water- | 0.5-5 | wt % |
| insoluble Polymer | | |
| Plasticizer(s) | 0-1 | wt % |
| Channeling Agents | 0.2-5 | wt % |
| Overcoat: | | |
| Enteric Polymer | 0-30 | wt % |
| Anti-sticking Agent | 0-6 | wt % |
| Plasticizer | 0-6 | wt % |
| Channeling Agents | 0-6 | wt % |
| TOTAL | 100 | wt % |

A particularly preferred tablet which is useful in the practice of the invention has the ingredients as set forth in Table 2 and may be prepared as set forth below:

TABLE 2

| | | | | |
|---|---|---|---|---|
| Lovastatin | 12.14 | wt % | 20.00 | mg |
| Polyox WSR Coagulant, NF (polyethylene | 4.55 | wt % | 7.50 | mg |
| oxide Mw No. AV 5,000,000) | | | | |
| Polyox WSR N 80, NF (polyethylene oxide | 17.76 | wt % | 29.25 | mg |
| Mw No. AV 200,000) | | | | |
| Lactose (anhydrous) | 51.30 | wt % | 84.50 | mg |
| Sodium lauryl sulfate | 3.04 | wt % | 5.00 | mg |
| Cab-O-Sil (Silicon dioxide Fumed US/NF) | 0.46 | wt % | 0.75 | mg |
| Butylated hydroxy anisole | 0.03 | wt % | 0.05 | mg |
| Myvaplex 600P (glyceryl monostearate) | 1.82 | wt % | 3.00 | mg |
| Seal Coating: | | | | |
| Opadry Clear (mixture containing | 3.42 | wt % | 5.63 | mg |
| hydroxypropyl methyl cellulose and | | | | |
| polyethylene glycol) | | | | |
| Sodium chloride | 1.14 | wt % | 1.88 | mg |
| Outer Coating: | | | | |
| Cellulose acetate | 1.43 | wt % | 2.36 | mg |
| Eudragit S 100 (poly(methylacrylic acid) | 0.49 | wt % | 0.80 | mg |
| methylacrylate) 1:2 ratio MW (No. Av. | | | | |
| 135,000 - USP Type B) | | | | |
| Triacetin (Glycerol Triacetate) | 0.11 | wt % | 0.19 | mg |
| Polyethylene glycol 400 | 0.11 | wt % | 0.19 | mg |
| Sugar, confectioners 6X micronized | 0.72 | wt % | 1.18 | mg |
| Overcoat: | | | | |
| Hydroxypropylmethylcell. Phthal. 55 | 0.77 | wt % | 1.27 | mg |
| Talc | 0.30 | wt % | 0.49 | mg |
| Acetyl tributyl citrate | 0.12 | wt % | 0.20 | mg |
| Sugar, confectioners 6X micronized | 0.30 | wt % | 0.49 | mg |
| | 100.0 | wt % | 146.73 | mg |

The following describes the preferred process of making the above described dosage form:

Step 1. The Tablet Core

(a) Granulation
1. Pass Polyox WSR N80, sodium lauryl sulfate and anhydrous lactose through a 30 mesh stainless steel screen.
2. Charge the screened materials and lovastatin (micronized) into a vertical granulator.
3. Prepare a butylated hydroxy anisole solution by dissolving butylated hydroxy anisole in ethanol.
4. Prepare a mixture of ethanol and purified water.
5. Pre-mix the powder mixture from above (step 1(a)2) for 5 minutes.
6. Blend the powder mixture again, add the butylated hydroxyanisole solution and then the ethanol/water mixture.
7. Dry the resulting granules at 45-50° C. until the moisture content is lower than 1.8 wt %.
8. Pass the granules through a 1575 mesh using a Comil.

(b) Tabletting
1. Mix Cab-O-Sil and Polyox WSR N80.
2. Pass the mixture of Cab-O-Sil and Polyox WSR N80 through a 24 mesh stainless steel screen with the Polyox WSR Coagulant.
3. Blend the screened materials with lovastatin granules for 15 minutes.
4. Pass Myvaplex through a 30 mesh stainless steel screen and combine with the other screen materials.
5. Blend for five minutes.
6. Compress the blend into tablets (164.72 mg, round, standard concave, 17/64″ dia.) which contain 20 mg of lovastatin.

(c) Seal Coating: Opadry Clear
1. Dissolve sodium chloride in purified water.
2. Disperse Opadry Clear into the sodium chloride solution.
3. Spray lovastatin tablets with the aqueous coating suspension using a coater.

(d) Inner Coating: None

(e) Outer Coating: Cellulose Acetate
1. Dissolve cellulose acetate and Eudragit S100 in acetone using a homogenizer.
2. Add polyethylene glycol 400, triacetein and sugar to the solution and mix until a homogenous dispersion is obtained.
3. Spray the coating suspension onto the tablets in a coater.

(f) Overcoating: Hydroxypropyl methylcellulose phthalene 55 (HPMCP 55)
1. Dissolve hydroxypropyl methylcellulose phthalene 55 in acetone using a homogenizer.
2. Add acetyl tributyl citrate to the acetone solution and mix it with a homogenizer until a homogenized dispersion is obtained.
3. Add talc and sugar to the solution and mix it with a homogenizer until a homogenized dispersion is obtained.
4. Replace the homogenizer with a magnetic mixer and stir the coating mixture throughout the coating process.
5. Spray the Opadry Clear coated lovastatin tablets with the coating dispersion in a coater.

Other particularly preferred control release tablets useful in the practice of the present invention are those disclosed in U.S. patent application Ser. No. 09/435,576, which is herein incorporated by reference.

For example, a particularly preferred tablet which is useful in the practice of the present invention has the ingredients as set forth in Table 3 and may be prepared as set forth below:

TABLE 3

| | | | | |
|---|---|---|---|---|
| Lovastatin | 11.99 | wt % | 40.0 | mg |
| Polyox WSR Coagulant, NF (polyethylene | 4.50 | wt % | 15.0 | mg |
| oxide Mw No. AV 5,000,000) | | | | |
| Polyox WSR N 80, NF (polyethylene | 17.98 | wt % | 60.0 | mg |
| oxide Mw No. AV 200,000) | | | | |
| Lactose (anhydrous) | 50.65 | wt % | 169.0 | mg |
| Sodium lauryl sulfate | 3.00 | wt % | 10.0 | mg |
| Silicon dioxide Fumed USP/NF | 0.45 | wt % | 1.5 | mg |
| Myvaplex 600P (glyceryl monostearate) | 1.80 | wt % | 6.0 | mg |
| Seal Coating: | | | | |
| Opadry Clear (mixture containing | 2.81 | wt % | 9.4 | mg |
| hydroxypropyl methyl cellulose and | | | | |
| polyethylene glycol | | | | |
| Sodium chloride | 0.93 | wt % | 3.1 | mg |
| Inner Coating: | | | | |
| Hydroxypropylmethylcell.phthal.55 | 2.27 | wt % | 7.58 | mg |
| Talc | 0.78 | wt % | 2.60 | mg |
| Acetyl tributyl citrate | 0.22 | wt % | 0.75 | mg |
| Sugar, confectioners 6X micronized | 0.62 | wt % | 2.08 | mg |
| Outer Coating: | | | | |
| Cellulose acetate | 1.00 | wt % | 3.32 | mg |
| Eudragit S 100 (poly(methacrylic acid), | 0.34 | wt % | 1.13 | mg |
| methylmetharcrylate, 1:2 ratio MW | | | | |
| (No. Av. 135,000 - USP Type B) | | | | |
| Triacetin (Glycol Triacetate) | 0.08 | wt % | 0.27 | mg |
| Polyethylene glycol 400 | 0.08 | wt % | 0.27 | mg |
| Sugar, confectioners 6X micronized | 0.50 | wt % | 1.66 | mg |
| TOTAL | 100.00 | wt % | 333.66 | mg |

The following describes a suitable process of making the above described dosage form:

Granulation

1. Pass Polyox WSR N80, sodium lauryl sulfate and anhydrous lactose through a 30 mesh stainless steel screen.

2. Charge the screened materials and lovastatin (micronized) into a vertical granulator.

3. Dissolve buylated hydroxy anisole in ethanol.

4. Mix ethanol, and purified water.

5. Pre-mix the powder mixture for 5 minutes.

6. Blend the powder mixture again, add the butylated hydroxyanisole solution and then the ethanol/water mixture.

7. Dry the granules at 45-50° C. until the moisture content is lower than 1.8 wt %.

8. Pass the granules through a 1575 mesh using a Comil.

Tabletting

1. Mix Cab-O-Sil and Polyox WSR N80.

2. Pass the mixture of Cab-O-Sil and Polyvox WSR N80 through a 24 mesh stainless steel screen with the Polyox WSR Coagulant.

3. Blend the screen materials with lovastatin granules for 15 minutes.

4. Pass Myvaplex through a 30 mesh stainless steel screen and combine with the other screen materials.

5. Blend for five minutes.

6. Compress the blend into tablets (300 mg, round standard concave, 11/32″) which contain 40 mg of lovastatin.

Seal Coating: Opadry Clear

1. Dissolve sodium chloride in purified water.

2. Disperse Opadry Clear into the sodium chloride solution.

3. Spray lovastatin tablets with the aqueous coating suspension using a coater.

Inner Coating: Hydroxypropyl methylcellulose phthalate 55

1. Dissolve hydroxypropyl methylcellulose phthalate 55 in acetone using a homogenizer.

2. Add acetyl tributyl citrate to the acetone solution and mix it with a homogenizer until a homogenized dispersion is obtained.

3. Add talc and sugar to the solution and mix it with a homogenizer until a homogenized dispersion is obtained.

4. Replace the homogenizer with a magnetic mixer and stir the coating mixture throughout the coating process.

5. Spray the Opadry Clear coated lovastatin tablets with the coating dispersion in a coater.

Outer Coating: Cellulose Acetate

1. Dissolve cellulose acetate and Eudragit S100 in acetone using a homogenizer.

2. Add polyethylene glycol 400, triactein and sugar to the solution and mix until a homogeneous dispersion is obtained.

3. Spray the coating suspension onto the tablets in a coater.

Another example of a particularly preferred tablet has the ingredients as set forth in Table 4:

TABLE 4

| | | | | |
|---|---|---|---|---|
| Lovastatin | 12.11 | wt % | 40.0 | mg |
| Polyox WSR Coagulant, NF (polyethylene | 4.54 | wt % | 15.0 | mg |
| oxide Mw No av 5,000,000) | | | | |
| Polyox WSR N 80, NF (polyethylene | 17.71 | wt % | 58.5 | mg |
| oxide Mw No av 200,000) | | | | |
| Lactose (anhydrous) | 51.13 | wt % | 168.9 | mg |
| Sodium lauryl sulfate | 3.03 | wt % | 10.0 | mg |
| Cab-O-Sil (Silicon dioxide Fumed | 0.45 | wt % | 1.5 | mg |
| USP/NF) | | | | |
| Butylated hydroxy anisole | 0.03 | wt % | 0.10 | mg |
| Myvaplex 600P (glyceryl monostearate) | 1.82 | wt % | 6.0 | mg |
| Seal Coating: | | | | |
| Opadry Clear (mixture containing | 2.85 | wt % | 9.4 | mg |
| hydroxypropyl methyl cellulose and | | | | |
| polyethylene glycol | | | | |
| Sodium Chloride | 0.94 | wt % | 3.1 | mg |
| Inner Coating: | | | | |
| Hydroxypropylmethylcell.phthal.55 | 2.29 | wt % | 7.58 | mg |
| Talc | 0.79 | wt % | 2.6 | mg |
| Acetyl tributyl citrate | 0.23 | wt % | 0.75 | mg |
| Sugar, confectioners 6X micronized | 0.08 | wt % | 0.27 | mg |
| Outer Coating: | | | | |
| Cellulose acetate | 1.00 | wt % | 3.32 | mg |
| Eudragit S 100 (polymethacrylicacid, | 0.34 | wt % | 1.13 | mg |
| methylmethaceylate, 1:2 ratio MW | | | | |
| (No. AV. 135,000 - USP Type B) | | | | |
| Triacetin | 0.08 | wt % | 0.27 | mg |
| Polyethylene glycol 400 | 0.08 | wt % | 0.27 | mg |
| Sugar, confectioners 6X micronized | 0.50 | wt % | 1.66 | mg |
| TOTAL | 100.00 | wt % | 330.35 | mg |

The preferred tablet having the ingredients as set forth in Table 4 may be prepared as described above for the preparation of the preferred tablet having the ingredients as set forth in Table 3.

Another example of a particularly preferred tablet has the ingredients as set forth in Table 5 and may be prepared as set forth below:

TABLE 5

| | | | | |
|---|---|---|---|---|
| Lovastatin | 12.14 | wt % | 20.0 | mg |
| Polyox WSR Coagulant, NF | 4.55 | wt % | 7.5 | mg |
| (polyethylene oxide Mw No. AV | | | | |
| 5,000,000) | | | | |
| Polyox WSR N 80, NF (polyethylene | 17.76 | wt % | 29.25 | mg |
| oxide Mw No. AV 200,000) | | | | |
| Lactose (anhydrous) | 51.30 | wt % | 84.5 | mg |
| Sodium lauryl sulfate | 3.04 | wt % | 5.0 | mg |
| Cab-O-Sil (Silicon dioxide Fumed | 0.46 | wt % | 0.75 | mg |
| USP/NF) | | | | |
| Butylated hydroxy anisole | 0.03 | wt % | 0.05 | mg |
| Myvaplex 600P (glyceryl monostearate) | 1.82 | wt % | 3.0 | mg |
| Seal Coating: | | | | |
| Opadry Clear (mixture containing | 3.42 | wt % | 5.63 | mg |
| hydroxypropyl methyl cellulose and | | | | |
| polyethylene glycol) | | | | |
| Sodium chloride | 1.14 | wt % | 1.88 | mg |
| Outer Coating: | | | | |
| Cellulose acetate | 1.43 | wt % | 2.36 | mg |
| Eudragit S 100 (polymethylacrylic acid, | 0.49 | wt % | 0.80 | mg |
| methylacrylate, 1:2 ratio MW (No. Av. | | | | |
| 135,000 - USP Type B) | | | | |
| Triacetin | 0.11 | wt % | 0.19 | mg |
| Polyethylene glycol 400 | 0.11 | wt % | 0.19 | mg |
| Sugar, confectioners 6X micronized | 0.72 | wt % | 1.18 | mg |
| Overcoat: | | | | |
| Hydroxypropylmethylcell. Phthal. 55 | 0.77 | wt % | 1.27 | mg |
| Talc | 0.30 | wt % | 0.49 | mg |
| Triacetin | 0.12 | wt % | 0.20 | mg |
| Sugar, confectioners 6X micronized | 0.30 | wt % | 0.49 | mg |
| TOTAL | 100.0 | wt % | 0146.73 | mg |

The following describes the process of making the above described dosage form:

Granulation

1. Pass Polyox WSR N80, sodium lauryl sulfate and anhydrous lactose through a 30 mesh stainless steel screen.

2. Charge the screened materials and lovastatin (micronized) into a vertical granulator.

3. Dissolve butylated hydroxy anisole in ethanol.

4. Mix ethanol and purified water.

5. Pre-mix the powder mixture for 5 minutes.

6. Blend the powder mixture again, add the butylated hydroxyanisole solution and then the ethanol/water mixture.

7. Dry the granules at 45-50° C. until the moisture content is lower than 1.8 wt %.

8. Pass the granules through a 1575 mesh using a Comil.

Tabletting

1. Mix Cab-O-Sil and Polyox WSR N80.

2. Pass the mixture of Cab-O-Sil and Polyox WSR N80 through a 24 mesh stainless steel screen with the Polyox WSR Coagulant.

3. Blend the screen materials with lovastatin granules for 15 minutes.

4. Pass Myvaplex through a 30 mesh stainless steel screen and combine with the other screen materials.

5. Blend for five minutes.

6. Compress the blend into tablets (164.72 mg, round, standard concave, 17/6411dia.) which contain 20 mg of lovastatin.

Seal Coating: Opadry Clear

1. Dissolve sodium chloride in purified water.

2. Disperse Opadry Clear into the sodium chloride solution.

3. Spray lovastatin tablets with the aqueous coating suspension using a coater.

Inner Coating: None

Outer Coating: Cellulose acetate

1. Dissolve cellulose acetate and Eudragit S100 in acetone using a homogenizer.

2. Add polyethylene glycol 400, triactein and sugar to the solution and mix until a homogeneous dispersion is obtained.

3. Spray the coating suspension onto the tablets in a coater.

Overcoating: Hydroxypropyl methylcellulose phthalate 55

1. Dissolve hydroxypropyl methylcellulose phthalate 55 in acetone using a homogenizer.

2. Add acetyl tributyl citrate to the acetone solution and mix it with a homogenizer until a homogenized dispersion is obtained.

3. Add talc and sugar to the solution and mix it with a homogenizer until a homogenized dispersion is obtained.

4. Replace the homogenizer with a magnetic mixer and stir the coating mixture throughout the coating process.

5. Spray the Opadry Clear coated lovastatin tablets with the coating dispersion in a coater.

Another example of a particularly preferred tablet has the ingredients as set forth in Table 6 and may be prepared by the same general procedure as described above for the preparation of the tablet having the ingredients as set forth in Table 5, except that no inner coating is applied and an outer enteric coating is applied as an overcoat over the outer layer.

TABLE 6

| | | | | |
|---|---|---|---|---|
| Lovastatin | 12.20 | wt. % | 20.0 | mg. |
| Polyox WSR Coagulant, NF | 4.57 | wt. % | 7.5 | mg. |
| (Polyethylene oxide Mw No av | | | | |
| 5,000,000) | | | | |
| Polyox WSR N 80, NF (polyethylene | 17.84 | wt/% | 29.25 | mg. |
| oxide Mw No av 200,000) | | | | |
| Lactose (anhydrous) | 51.53 | wt. % | 84.5 | mg. |
| Sodium lauryl sulfate | 3.05 | wt. % | 5.0 | mg. |
| Silicon dioxide fumed USP/NF | 0.46 | wt. % | 0.75 | mg. |
| Butylated hydroxy anisole | 0.03 | wt. % | 0.05 | mg. |
| Myvaplex 600P (glyceryl monostearate) | 1.83 | wt. % | 3.0 | mg. |
| Seal Coating: | | | | |
| Opadry Clear (mixture containing | 3.43 | wt. % | 5.63 | mg. |
| hydroxypropyl methyl cellulose and | | | | |
| polyethylene glycol) | | | | |
| Sodium chloride | 1.15 | wt. % | 1.88 | mg. |
| Inner Coating: None | | | | |
| Outer Coating: | | | | |
| Cellulose acetate | 1.96 | wt. % | 3.21 | mg. |
| Eudragit S 100 | 0.66 | wt. % | 1.09 | mg. |
| Acetyl tributyl citrate | 0.32 | wt. % | 0.52 | mg. |
| Sugar, confectioners 6X micronized | 0.98 | wt. % | 1.61 | mg. |
| TOTAL | 100.00 | wt. % | 163.99 | mg. |

Other examples of particularly preferred 40 mg. tablets have the ingredients as set forth in Table 7 and may be prepared by the same method described above for preparing the tablet having the ingredients as set forth in Table 3.

TABLE 7
Summary of Lovastatin Formulations

| | Weight Percent | | | | | |
|---|---|---|---|---|---|---|
| Ingredient | Tablet A | | Tablet B | | Tablet C | |
| Lovastatin (strength, mg) | 40 | wt. % | 40 | mg. | 40 | mg. |
| Tablet Core | | | | | | |
| 1. Lovastatin | 12.11 | wt. % | 12.28 | wt. % | 12.28 | wt. % |
| 2. Lactose (Anhydrous) | 51.13 | wt. % | 51.8 | wt. % | 51.8 | wt. % |
| 3. Polyox ® WSR Coagulant | 4.54 | wt. % | 4.6 | wt. % | 4.6 | wt. % |
| 4. Polyox ® WSR N80 | 17.71 | wt. % | 17.94 | wt. % | 17.94 | wt/% |
| 5. Sodium Lauryl Sulfate | 3.03 | wt. % | 3.06 | wt. % | 3.06 | wt. % |
| 6. Glyceryl Monostearate | 1.82 | wt. % | 1.84 | wt. % | 1.84 | wt. % |
| 7. Silicon Dioxide | 0.45 | wt. % | 0.46 | wt. % | 0.46 | wt. % |
| 8. Butylated Hydroxyanisole | 0.03 | wt. % | 0.02 | wt. % | 0.02 | wt. % |
| Seal Coat | | | | | | |
| 1. Opadry Clear | 2.85 | wt. % | 2.88 | wt. % | 2.88 | wt. % |
| 2. Sodium Chloride Powder | 0.94 | wt. % | 0.96 | wt. % | 0.96 | wt. % |
| Inner Coat | | | | | | |
| 1. HPMCP 55 | 2.29 | wt. % | 1.61 | wt. % | 1.61 | wt. % |
| 2. Talc, USP | 0.79 | wt. % | 0.55 | wt. % | 0.55 | wt. % |
| 3. Acetyltributyl Citrate | 0.23 | wt. % | 0.16 | wt. % | 0.16 | wt. % |
| 4. Sugar, Micronized | 0.64 | wt. % | 0.44 | wt. % | 0.44 | wt. % |
| Outer Coat | | | | | | |
| 1. Cellulose Acetate | 1 | wt. % | 0.7 | wt. % | 0.7 | wt. % |
| 2. Eudragit S100 | 0.34 | wt. % | 0.24 | wt. % | 0.24 | wt. % |
| 3. Triacetin | 0.08 | wt. % | 0.06 | wt. % | 0.06 | wt. % |
| 4. Polyethylene Glycol 400 | 0.08 | wt. % | 0.6 | wt. % | 0.6 | wt. % |
| 5. Acetyltributyl Citrate | — | | — | | — | |
| 6. Sugar, Micronized | 0.5 | wt. % | 0.35 | wt. % | 0.35 | wt. % |
| Overcoat | | | | | | |
| 1. HPMCP 55 | — | | — | | — | |
| 2. Talc, USP | — | | — | | — | |
| 3. Triacetin | — | | — | | — | |
| 4. Sugar, Micronized | — | | — | | — | |
| 5. Opadry Yellow | — | | — | | — | |
| 6. Opadry Pink | — | | — | | — | |
| TOTAL TABLET | 100 | wt. % | 100 | wt. % | 100 | wt. % |
| WEIGHT, % | | | | | | |

Examples of other preferred tablets having the ingredients as set forth in Table 8 may be prepared by the same method described above for preparing the tablet having the ingredients as set forth in Table 3.

TABLE 8
Summary of Lovastatin Formulations

Weight Percent

| Ingredient | Tablet D | | Tablet E | |
|---|---|---|---|---|
| Lovastatin (strength, mg) | 20 | mg. | 10 | mg. |
| Tablet Core | | | | |
| 1. Lovastatin | 11.69 | wt. % | 5.84 | wt. % |
| 2. Lactose (Anhydrous) | 49.32 | wt. % | 55.18 | wt. % |
| 3. Polyox ® WSR Coagulant | 4.38 | wt. % | 4.38 | wt. % |
| 4. Polyox ® WSR N80 | 18.08 | wt. % | 17.09 | wt. % |
| 5. Sodium Lauryl Sulfate | 2.92 | wt. % | 2.92 | wt. % |
| 6. Blyceryl Monostearate | 1.75 | wt. % | 1.75 | wt. % |
| 7. Silicon Dioxide | 0.44 | wt. % | 0.44 | wt. % |
| 8. Butylated Hydroxyanisole | 0.02 | wt. % | 0.01 | wt. % |
| Seal Coat | | | | |
| 1. Opadry Clear | 2.74 | wt. % | 2.74 | wt. % |
| 2. Sodium Chloride Powder | 0.91 | wt. % | 0.91 | wt. % |
| Inner Coat | | | | |
| 1. HPMCP 55 | 2.21 | wt. % | 2.21 | wt. % |
| 2. Talc, USP | 0.76 | wt. % | 0.76 | wt. % |
| 3. Acetyltributyl Citrate | 0.22 | wt. % | 0.22 | wt. % |
| 4. Sugar, Micronized | 0.61 | wt. % | 0.61 | wt. % |
| Outer Coat | | | | |
| 1. Cellulose Acetate | 0.97 | wt. % | 0.97 | wt. % |
| 2. Eudragit S100 | 0.33 | wt. % | 0.33 | wt. % |
| 3. Triacetin | 0.08 | wt. % | 0.08 | wt. % |
| 4. Polyethylene Glycol 400 | 0.08 | wt. % | 0.08 | wt. % |
| 5. Acetyltributyl Citrate | — | | — | |
| 6. Sugar, Micronized | 0.49 | wt. % | 0.49 | wt. % |
| Overcoat | | | | |
| 1. HPMCP 55 | — | | — | |
| 2. Talc, USP | — | | — | |
| 3. Triacetin | — | | — | |
| 4. Sugar, Micronized | — | | — | |
| 5. Opadry Yellow | 3 | wt. % | 3 | wt. % |
| 6. Opadry Pink | — | | — | |
| TOTAL TABLET WEIGHT, % | 100 | wt. % | 100 | wt. % |

As illustrated in the following examples, cholesterol depletion may lead to a decrease in the release and formation of Aβ peptides in the cells. Additionally, the applicants have discovered that the decreased release of Aβ peptides is not due to the accumulation of the Aβ peptide in the cells, but rather due to the decreased formation of Aβ peptides. Further, the formation APPₛ, is also reduced by cholesterol treatment with a HMG-CoA reductase inhibitor, but to a much lesser degree. Further, decreased maturation (glycosylation and sulfation) of APPᵢ has been excluded as a cause for the effects cholesterol depletion treatment with a HMG-CoA reductase inhibitor on APPₘ processing and Aβ peptide formation. Thus, applicants have discovered that reducing cellular cholesterol by the use of an HMG-CoA reductase inhibitor regulates APPₘ processing and Aβ formation.

In the following examples, EasyTag™ EXPRESS™ Methionine Protein Labeling Mix, [³⁵S] (spec. activity>1,000 Ci/mMol) was obtained from NEN Life Sciences, Boston, Mass.; Fetal Calf Lipid Depleted Serum (FCLPDS) was obtained from Intracel, Rockville, Md.; Dulbecco's modified Eagles Medium (DMEM) was obtained form BioWittaker, Walkersville, Md.; Dulbecco's phosphate buffered saline (PBS) and Fetal Bovine Serum (FBS) were obtained from Life Technologies, Rockville, Md.; Antibody 6E10 was obtained from Senetek, Napa, Calif. ; Agarose bound antisera anti-mouse IgG was obtained from American Qualex Antibodies, San Clemente, Calif.; Protein A sepharose was obtained from Pharmacia Biotech, Piscataway, N.J.; Tissue culture plates were obtained from Falcon, Lincoln Park, N.J. with the exception of the 10 mm culture dishes with glass coverslips which were obtained from MatTek Corporation, Ashland, N.Mex.; and all other chemicals were obtained from Sigma, St. Louis, Mo.

The three cell lines utilized were: Chinese Hamster Ovary (CHO) cells expressing the 751 amino acid form of APP; Mabin-Darby Canine Kidney (MDCK) cells which overexpress the 695 amino acid form of APP; Human neuroglioma (H4) cells overexpressing the 695 form of human APP. All cells were prepared by the stable introduction of a cDNA coding for human APP. All cell lines were maintained in DMEM containing 10% FBS and antibiotics.

### EXAMPLES

The following examples are intended to illustrate but not to limit the invention.

### Example 1

Effects of Cholesterol Depletion

In order to characterize the effects of cholesterol depletion, cell cultures of each cell line were cultured on six-well plates for 4 days in DMEM containing 10% FCLPDS, which lipid depleted medium reduced the external source of cholesterol, in the presence of lovastatin acid (LA) or absence of LA.

In order to confirm that this treatment was sufficient to reduce cellular cholesterol, filipin, a fluorescent dye that binds to cholesterol, was utilized to provide a visual and quantitative measure of the level of cholesterol in the membrane. Generally, the cells were plated onto 10 mm culture dishes in media containing DMEM with 10% FCLPDS and antibiotics. Following incubation, cells were washed once with PBS and fixed with 3% paraformaldehyde in PBS for 1 hour, followed by washing 3 times in PBS for 5 minutes and quenching with 1.5 mg/ml glycine in PBS for 10 minutes. The cells were subsequently stained with 0.5 mg/ml filipin in PBS for 2 hours and washed 3 times for 5 minutes in PBS. After the final wash, the cells were visualized under a fluorescent microscope.

For measurement of APP processing and Aβ peptide formation, medium was removed and the cells were washed once with PBS and then incubated for 2 hours in DMEM containing 1 mCi/ml [³⁵S] Methionine. After this “pulse” period, the cells were either (1) lysed to measure the total labeled APPᵢ and APPₘ at time zero, or (2) the cells were incubated for 2 hours in fresh, unlabeled complete medium (“chase”) and then lysed. Then the cell supernatants and lysates were treated with the appropriate antibody to calculate the amounts of APPᵢ, APPₘ, APPₛ, and Aβ peptides.

To measure cell-associated, full-length APPᵢ and APPₘ, or to measure carboxyl-terminal fragments of APP, cell lysates were incubated with antibody 369 which recognizes the carboxyl-terminus of APP. See Buxbaum, J. D., et al. (1990) Proc Natl Acad Sci USA 87:6003-6, which is incorporated herein by reference.

To measure Aβ peptides, or to measure APPₛ, which is the secreted carboxyl-terminal truncated form, cell supernatants were incubated with antibody 6E10, which recognizes the first 15 amino acids of the Aβ peptide that correspond to the COOH-terminal amino acids of APPₛ . See Buxbaum, J. D., et al. (1994) Proc Natl Acad Sci USA. 91:4489-93, which is incorporated herein by reference.

The incubations with antibody 6E10 or antibody 369 were performed at 4° C. for 75 minutes followed by a 45 minute incubation at 4° C. with either agarose-linked anti-mouse IgG for antibody 6E10 or protein A sepharose for antibody 369. The beads were then washed three times for 10 minutes and then run on either a 10-20% Tris-Tricine Gel for APPₛ and Aβ peptides or an 8% polyacrylamide gel for cell-associated APR The gels were dried and exposed to a Phosphor Imager® screen (STORM 860, Molecular Dynamics) and exposed for a minimum of two days. The protein bands were visualized on a STORM 860 Phosphor Imager® (Molecular Dynamics) and quantitated using ImageQuant® (Molecular Dynamics).

To measure extracellular APPₛ and fragments thereof, the cell culture supernatant were utilized. To measure the intracellular APPᵢ and APPₘ and fragments thereof, the cell lysates were utilized.

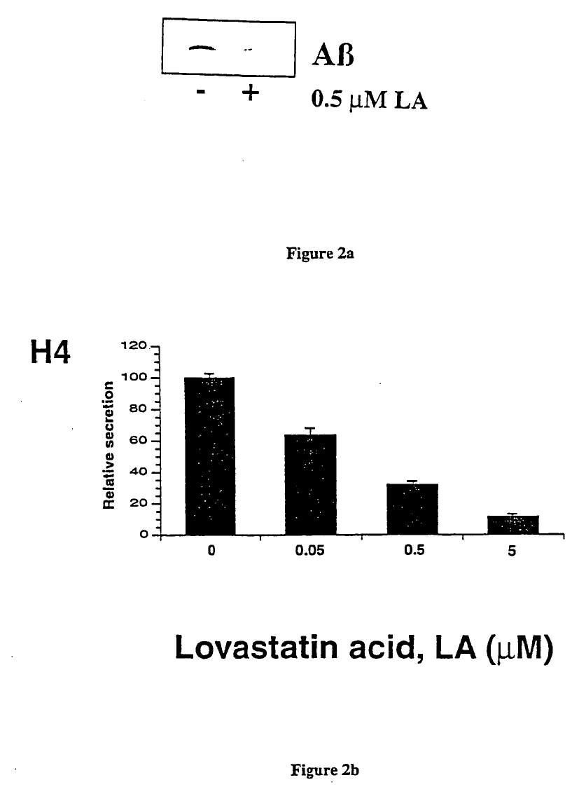

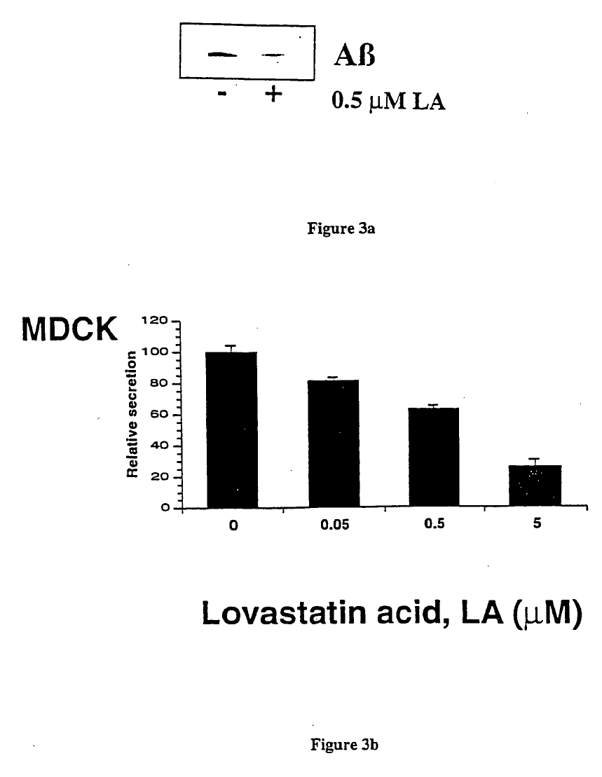

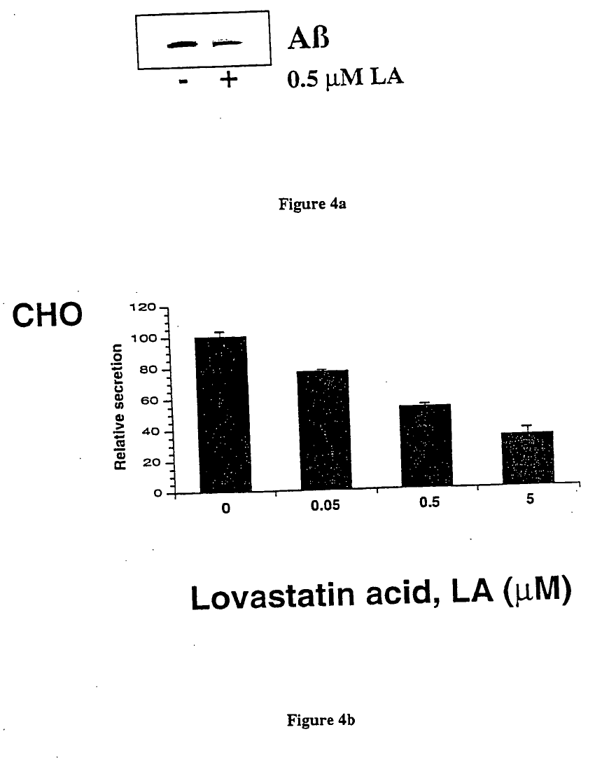

To determine whether a decrease in cholesterol is associated with a change in the amount of extracellular Aβ peptides, the H4, MDCK, and CHO cells, expressing human APPₘ were incubated for 4 days in the presence or absence of 0.5 μM LA. The cells were then incubated in serum-free media containing 1 mCi/Ml [³⁵S] Methionine for 2 hours followed by incubation in complete fresh serum-free medium containing unlabeled methionine for an additional 2 hours. The [³⁵S]-labeled Aβ peptides were immunoprecipitated from the cell culture supernatant, resolved by SDS-PAGE, and visualized by autoradiography. See FIGS. 2a, 3a, and 4a. Relative levels of extracellular Aβ peptides were determined under each condition by quantitative Phosphorlmager autoradiography. See FIGS. 2b, 3b, and 4b.

By subsequently focusing only on the [³⁵S]-labeled protein, the amounts of [³⁵S]-labeled Aβ peptides were normalized to the levels of total [³⁵S]-labeled APPᵢ by dividing total [³⁵S]-labeled Aβ by total [³⁵S]-labeled APPᵢ to exclude any changes in Aβ peptide levels due to decreased synthesis of Aβ peptides. At the end of the 2 hour chase the analysis was restricted to the proteolysis of APPₘ and secretion of Aβ peptides. See Buxbaum, J. D., et al. (1990) Proc Natl Acad Sci USA. 87:6003-6, which is herein incorporated by reference.

As shown in FIGS. 2a, 2b, 3a, 3b, 4a, and 4b, the amounts of extracellular Aβ peptides in the presence of 0.5 μM LA decreased by 40-60% as compared to the untreated cells. Treatment of cells with 0.5 μM LA had a weaker effect on decreasing extracellular Aβ peptide levels (<20% reduction).

To determine whether the decreased levels of extracellular Aβ peptides observed were due to decreased formation of Aβ peptides, rather than decreased secretion of Aβ peptides from the cells, the levels of [³⁵S]-labeled intracellular Aβ peptides within the cell were measured in the cell lysates. No detectable levels of intracellular Aβ peptides were observed in cells incubated in the presence or absence of 0.5 μM LA. Therefore, the decrease in extracellular Aβ peptides was not due to decreased secretion of Aβ peptides, but instead confirms that it was due to the decreased formation of Aβ peptides from the cleavage of APPₘ.

To determine whether cholesterol depletion affects other aspects of APP processing, H4 cells were incubated for four days in the presence or absence of 0.5 μM LA, and subjected to metabolic labeling. The levels of [³⁵S]-labeled APPᵢ and APPₘ were determined by irnmunoprecipitation from cell lysates with an antibody against the COOH-terminal of APP, followed by quantitative autoradiography. Similarly, the levels of [³⁵S]-labeled extracellular and intracellular Aβ peptides were determined by immunoprecipitation of either cell culture supernatants (extracellular Aβ peptides) or cell lysates (intracellular Aβ peptides). The amounts of each were normalized to the levels of [³⁵S]-labeled APPᵢ found in cells at the beginning of the chase. Normalization was done by dividing the relevant value by the levels of [³⁵S]-labeled APPᵢ found in cells at the beginning of the chase.

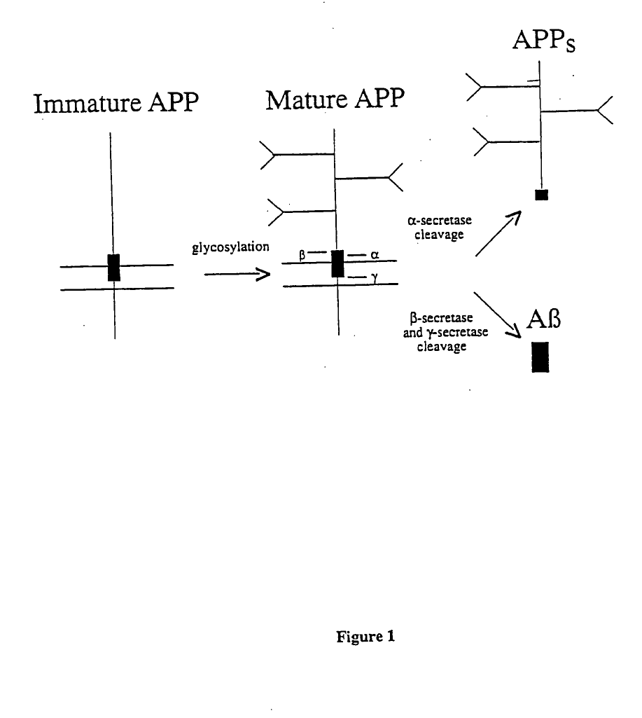

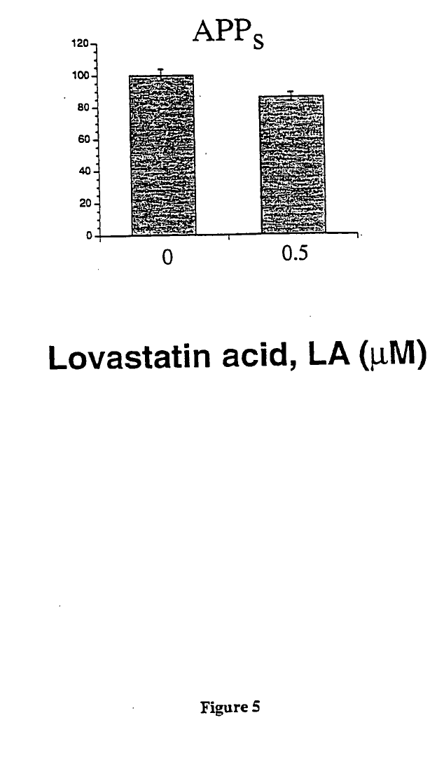

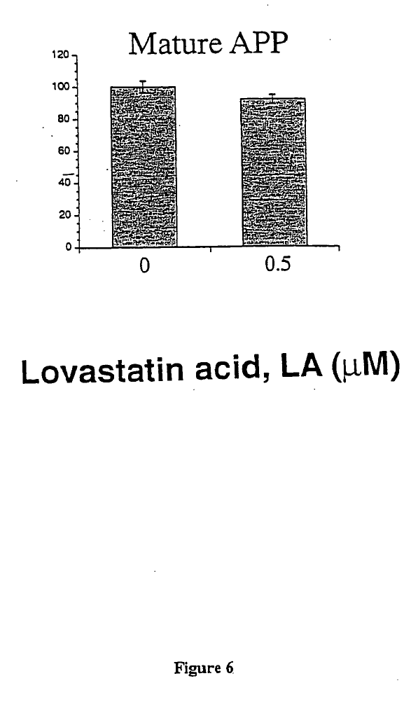

As illustrated in FIG. 5, a modest decrease in [³⁵S]-labeled APP, formation in cells incubated in the presence of LA, as compared to control cells was observed. FIG. 1 is a schematic illustrating APP processing. Because APPₘ is likely to be the precursor for both APPₛ and Aβ peptides, decreased formation of both APP, and Aβ peptides might suggest a decrease in the levels of APPₘ. To examine this, the levels of [³⁵S]-labeled APPₘ was measured in cells incubated in the absence or presence of 0.5 μM LA. As shown in FIG. 6, the effects on maturation were not sufficient to account for the decrease in Aβ peptide levels. Therefore, the effects of LA on Aβ peptide levels could not be accounted for by decreased maturation of APPₘ and instead reflect effects of LA on the post-Golgi processing or trafficking of APPₘ or both.

### Example 2

Effective Concentration Range of Lovastatin Acid

To determine the effective range of concentrations of LA, each cell type was grown in the absence or presence of various concentrations of LA by the methods described in Example 1. As shown in the bar graphs in FIGS. 2a, 2b, 3a, 3b, 4a, and 4b, the amount of extracellular Aβ peptides decreased with increasing LA concentrations and a concentration of 0.05 μM LA or higher was sufficient to significantly (p<0.001) decrease the amount of extracellular Aβ peptides under these experimental conditions.

### Example 3

Candidate Substance Screening

CHO cells were determined to be suitable for candidate screening because treatment with LA does not affect the maturation of APPᵢ to APPₘ in the CHO cells. Specifically, it was determined that treatment of CHO cells with 0.5 μM LA reduced the amount of extracellular APPₛ by about 30% of the amount calculated for the control and reduced the amount of extracellular Aβ peptides by about 70% of the amount calculated for the control when no LA was present. The amount of extracellular Aβ peptides and the amount of extracellular APPₛ were normalized to the amount of total APP found in the cell at the end of cell labeling as described above. This normalization provides an effective means of accounting for any differences between cultures and any differences due to altered APPᵢ synthesis or maturation in cells treated with the candidate compounds. However, the level of total APPₘ was comparable between the control and the treated cells (3.2×10⁶ arbitrary units and 3.4×10⁶ arbitrary units, respectively). This suggests that the maturation of APPᵢ to APPₘ was not affected in the CHO cells under experimental conditions.

Thus, CHO cells and other cells which manufacture Aβ peptides may be used as a suitable screening tool for a candidate substance which affects the synthesis, maturation or post-translational processing of APP. The cells are cultured on six-well plates for 4 days in DMEM containing 10% FCLPDS, which lipid depleted medium reduced the external source of cholesterol, in the presence of the candidate substance or absence of the candidate substance.

Specifically, the CHO cells are pulsed with [³⁵S] Methionine in the absence and presence of the candidate substance. After the pulse period, the cells are either (1) chased for two hours, or (2) lysed to determine the total intracellular APP at time zero.

Then the lysates may be labeled with the appropriate antibody to calculate the amounts of APPᵢ, APPₘ, APPₛ, and Aβ peptides.

To measure cell-associated, full-length APPᵢ and APPₘ, or to measure carboxyl-terminal fragments of APP, cell lysates are incubated with antibody 369 which recognizes the carboxyl-terminus of APP. See Buxbaum, J. D., et al. (1990) Proc Natl Acad Sci USA 87:6003-6, which is incorporated herein by reference.

To measure Aβ peptides, or to measure APPₛ , which is the secreted carboxyl-terminal truncated form, cell supernatants are incubated with antibody 6E10, which recognizes the first 15 amino acids of the Aβ peptide that correspond to the COOH-terminal amino acids of APPₛ, See Buxbaum, J. D., et al. (1994) Proc Natl Acad Sci USA. 91:4489-93, which is incorporated herein by reference.

The incubations with antibody 6E10 or antibody 369 are performed at 4° C. for 75 minutes followed by a 45 minute incubation at 4° C. with either agarose-linked anti-mouse IgG for antibody 6E10 or protein A sepharose for antibody 369. The beads are then washed three times for 10 minutes and then run on either a 10-20% Tris-Ticine Gel for APPₛ and Aβ peptides or an 8% polyacrylamide get for cell-associated APP. The gels are dried and exposed to a Phosphor Imager® screen and exposed for a minimum of two days. The protein bands are visualized on a STORM 860 Phosphor Imager® (Molecular Dynamics) and quantitated using ImageQuant (Molecular Dynamics).

To measure extracellular APPₛ and fragments thereof, the cell culture supernatant are utilized. To measure the intracellular APPi and APPₘ and fragments thereof, the cell lysates are utilized.

The amount of extracellular Aβ peptides and the amount of extracellular APPₛ are normalized to the amount of total APPᵢ found in the cell at the end of cell labeling as described above. This normalization provides an effective means of accounting for any differences between cultures and any differences due to altered APPᵢ synthesis or maturation in cells treated with the candidate compounds.

### Example 4

Human Trials

A study was conducted to assess the effects of Lovastatin XL on blood lipid levels in patients with hyperlipidemia. Patients were treated with placebo, 10, 20, 40 or 60 mg per day of lovastatin administered as Lovastatin XL. Blood samples were obtained from selected patients prior to dosing and at 1 month after dosing. Because this clinical trial was carried out as part of an New Drug Application, at the time of filing U.S. Provisional Patent Application No. 60/223,987 (“the '987 application”), the inventors were not permitted to determine what dose (placebo, 10, 20, 40 or 60 mg) each of the selected patients where given per day. These blood samples were assayed for Aβ peptide concentrations (pg/ml). The results are listed in Table 9 below:

TABLE 9
Baseline and 1-Month Beta Amyloid Values

| | Baseline Aβ peptide | Change in Aβ peptide |
|---|---|---|
| Patient | conc. (pg/ml) | conc. after 1 Month (pg/ml) |
| 1 | 145.2 | −57.8 |
| 2 | 211.1 | −30.5 |
| 3 | 151.1 | −16.9 |
| 4 | 175.5 | 60.6 |
| 5 | 388.1 | 44 |
| 6 | 499.7 | −172.7 |
| 7 | 164 | −64.1 |
| 8 | 220 | −67.5 |
| 9 | 215.5 | −80.3 |
| 10 | 370.1 | −18.5 |
| 11 | 403.9 | −76.2 |
| 12 | 48.9 | −32.9 |
| 13 | 15.6 | 38.4 |
| 14 | 64.6 | −13.4 |
| 15 | 34.3 | −18 |
| 16 | 12 | −1.1 |
| 17 | 45.4 | 10.9 |
| 18 | 12 | 4.6 |
| 19 | 37.3 | −5.6 |
| 20 | 30.6 | 0.2 |
| 21 | 35.2 | 10 |
| 22 | 138.5 | −112.5 |
| 23 | 60.6 | −38.5 |
| 24 | 50 | 22 |
| 25 | 73 | −22 |
| 26 | 133 | −17 |
| 27 | 23 | 70 |
| 28 | 82 | −21 |
| 29 | 4 | 4 |
| 30 | 56 | 5 |
| 31 | 59 | 17 |
| 32 | 2 | 4 |
| 33 | 3 | 1 |
| 34 | 181 | −42 |
| 35 | 0 | 5 |
| 36 | 175.4 | −37 |
| Mean | 120.016667 | −18.0222222 |
| SD | 126.784261 | 46.9449472 |

As can be seen from Table 9, the mean Aβ peptide concentration prior to treatment was 120 pg/ml, which decreased by about 18 pg/ml after one month of treatment with Lovastatin XL. The change from pre-treatment was statistically significant (p=0.0273) as shown in Table 10. In the instant case, one of ordinary skill in the art will understand that the above referred to p value represents the probability that the reported change in Aβ peptide concentration could occur by chance. One of ordinary skill in the art will also understand that a p value of less that 0.05 signifies that the reported change is statistically meaningful.

TABLE 10
Statistical Analysis of Change From Baseline
Results:
Two tailed T-test results:

| Variable | MU0 | Estimate | Std. Err, | DF | Tstat | Pval |
|---|---|---|---|---|---|---|
| var1 | 0 | −18.022223 | 7.8241577 | 35 | −2.3034072 | 0.0273 |

Following the filing of the '987 application, the inventors have performed a more detailed analysis of the data obtained from the human trials. Table 11 sets forth a dose-response analysis of the data and shows the mean percentage change in the Aβ peptide concentration in the blood of the patients treatment with placebo, 10, 20, 40 and 60 mg/day Lovastatin XL. Blood samples were taken from the patients after four weeks of treatment (Study Visit No. 5), after eleven weeks of treatment (Study Visit No. 7) and twelve weeks of treatment with Lovastatin XL (Study Visit No. 8).

TABLE 11
Effect of Various Doses of Lovastatin XL on Aβ Peptide Concentration

| | | Study | | Average of | |
|---|---|---|---|---|---|
| DOSE | | Visit | Study Visit | Study Visit | |
| (mg/day) | | No. 5 | No. 7 | Nos. 7 & 8 | ENDPOINT |
| 0 | N | 11 | 6 | 8 | 12 |
| | MEAN | −1.78 | −16.27 | −21.11 | −11.87 |
| | STD | 33.69 | 29.03 | 23.16 | 27.03 |
| 10 | N | 10 | 5 | 6 | 10 |
| | MEAN | 8.09 | 43.29 | 10.21 | −0.21 |
| | STD | 64.92 | 72.69 | 57.83 | 49.66 |
| 20 | N | 10 | 5 | 8 | 11 |
| | MEAN | 0.59 | −18.21 | −27.81 | −21.71 |
| | STD | 35.56 | 43.00 | 49.91 | 45.04 |
| 40 | N | 6 | 3 | 4 | 10 |
| | MEAN | −6.51 | −6.15 | −26.35 | −11.92 |
| | STD | 22.75 | 32.05 | 48.13 | 46.12 |
| 60 | N | 8 | 3 | 7 | 10 |
| | MEAN | −5.08 | −16.27 | −35.47 | −39.42 |
| | STD | 39.78 | 25.78 | 27.83 | 30.09 |

N—No. of patients
MEAN—mean % change in the Aβ peptide concentration in the blood of patients
STD—Standard deviation

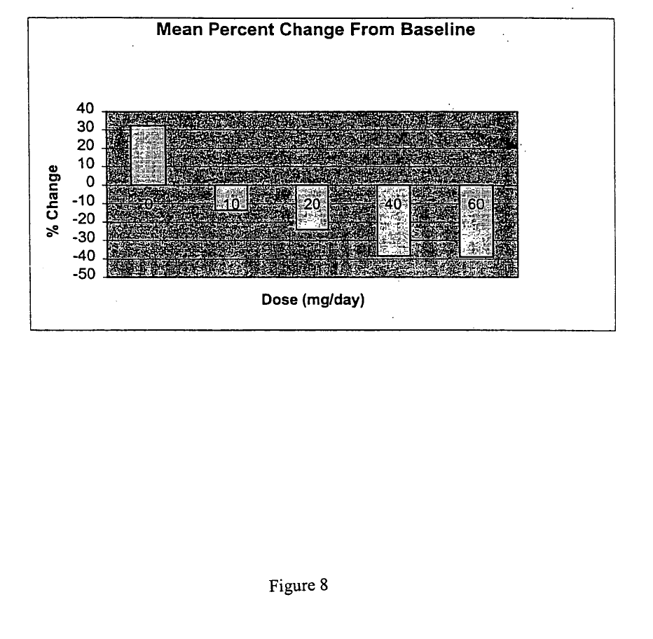

The graph shown in FIG. 8 depicts the results set forth in Table 11 above. In particular, the graph shows the change in mean Aβ peptide concentration in the blood of patients after one month of treatment with Lovastatin XL as a function of the dose administered. FIG. 8 also includes a “trendline”, i.e., the best straight-line approximation, of the data presented in the graph. As can be seen from the “trendline”, its direction and slope clearly suggests that the dose of Lovastin XL administered does have an effect on the mean Aβ peptide concentration. The inventors also found that the dose-response analysis for the endpoint values was statistically significant (p=0.0442).

Further human trials have been conducted. Patients meeting the current criteria for treatment with lipid lowering agents were treated with single-blind placebo for 4 weeks. Those patients were then randomly assigned to receive daily. doses of 10, 20, 40 or 60 mg/day of a controlled release lovastatin (Lovastatin XL) or matching placebo under double-blind conditions. Serum samples from those patients were obtained prior to and after 3 months of dosing. The serum samples were assayed for Aβ peptide using the assay set forth in Example 5 below. The assay results, expressed as percent change from pre-treatment of serum Aβ peptide concentration levels, are shown in FIG. 9.

As can be seen from FIG. 9, the placebo treated patients showed a mean increase of serum Aβ peptide concentration levels from baseline, however, this difference was not found to be statistically significant. The mean percent changes in the serum Aβ peptide concentration levels for the patients who received treatment with the controlled release formulation of lovastatin all decreased. These reported percentage changes for the groups of patients treated with 20, 40 and 60 mg/day were determined to be statistically significant p<0.01 (t-test). Further, the percentage changes for the groups of patients treated with 40 and 60 mg/day were determined to be statistically significantly different from those of the placebo group, p<0.05 (t-test).

### Example 5

AB End-Specific Protocol

The serum samples referred to above in Example 4 were assayed for Aβ peptide using appropriate assays. The assay used for the human trials, the results of which are set forth in FIG. 9, was carried out as follows:

Ninety-six well plates (Falcon Probind) were coated with 150 μl of the 4G8 monoclonal antibody (Senetek Crude IgG Ascites Fluid) in carbonate-bicarbonate buffered solution (Sigma) and then incubated at 37° C. for 12-16 hours. The plates were then washed three times with 150 μl/well ECW buffer (PBS, 0.1% BSA, 0.05% Tween-20, 0.2% CHAPS, 5mM ethylenediaminetetraacetic acid, 2 mM betaine, 0.05% NaN₃) before adding 150 μl/well ECW buffer containing 1% casein and incubated at 37° C. for an additional 4 hours. The 4G8 antibody recognizes Aβ and thus selects this peptide from the pool of others in the plasma.

The coated plates were washed twice with 150 μl/well ECW and then 50 μl/well ECW was added to ensure that the wells did not dry out during sample loading.

The standard curves of synthetic Aβ 1-40 peptide were prepared by diluting the 100 ng/μl into working solutions using ECW. For the assays the following concentrations were used: 0, 10, 50, 100, 250 and 500 pg/ml synthetic peptide. The standards were loaded in duplicate onto the wells.

Each plasma sample was thawed and then sonicated for 20 seconds prior to loading onto wells in quadruplicate. Each plate contained two internal reference samples and all patient visits were loaded on the same plate.

The loaded plates were incubated for 5 minutes at room temperature, and then for 2 days at 4° C. (“capture phase”). The plates were then washed twice with 150 μl/well ECW, 150 μl of biotinylated 6E10 monoclonal antibody (Senetek mAbs Biotin 6E10) diluted 1:1000 in ECW was added to each well and the plates incubated at room temperature for 12-15 hours. The 6E10 antibody recognizes Aβ which is “captured” by the 4G8 and is biotinylated so that it can be detected by the tertiary antibody.

The plates were washed three times with 150 μl/well ECW before 150 μl of streptavidin alkaline phophatase (Amersham) was added per well and incubated at room temperature for 5 hours. The plates were then washed three times with 150 μl/well ECW before 100 μl/well ddH₂O was added. The water was then aspirated and 100 μl of the Attophos reagent (JBL Scientific Inc) added per well before being allowed to develop at room temperature in the dark. When the highest point in the standard curve began to turn yellow, the plates were read on a microplate reader (PerSeptive Biosystems CytoFluor Series 4000) at an excitation of 450 run and an emission of 530 nm.

The foregoing description has been presented for purposes of illustration and description. It is not intended to be exhaustive or to limit the invention. The accompanying drawings are included to provide a further understanding of the invention and are incorporated in and constitute a part of this specification, illustrate several embodiments of the invention and together with the description serve to explain the principles of the invention. Obvious modifications or variations are possible in light of the above teachings. All such obvious modification and variations are intended to be within the scope of the present invention.

1-30. (canceled)

31. A method of treating Alzheimer's disease in humans, comprising
determining whether a human exhibits an elevated level of β-amyloid;
orally administering to a human patient found to exhibit an elevated level of β-amyloid a controlled release formulation comprising at least one HMG-CoA reductase inhibitor which after oral administration to a human patient releases said at least one HMG-CoA reductase inhibitor at a rate to maintain therapeutically effective levels over a 24 hour dosing interval, and
continuing treatment with said controlled release formulation to effect a decrease in mean beta amyloid concentration in the blood of said human patient by at least about 18 pg/ml after 1 month of treatment.

32. A method for treating Down's Syndrome in humans, comprising
orally administering to a human patient found to have Down's Syndrome a controlled release formulation comprising at least one HMG-CoA reductase inhibitor which after oral administration to a human patient releases said at least one HMG-CoA reductase inhibitor at a rate to maintain therapeutically effective levels over a 24 hour dosing interval, and
continuing treatment with said controlled release formulation to effect a decrease in mean beta amyloid concentration in the blood of said human patient by at least about 18 pg/ml after 1 month of treatment.

33. The method of claim 31, wherein therapy is continued for a minimum period of at least 90 days.

34. The method of claim 31, wherein therapy is continued for a minimum period of 90 to 365 days.

35. The method of claim 31, wherein said HMG-CoA reductase inhibitor is selected from the group consisting of mevastatin, pravastatin, simvastatin, atorvastatin, lovastatin, rivastatin, fluvastatin, and pharmaceutically acceptable salts, isomers, or metabolites thereof.

36. The method of claim 31, wherein said HMG-CoA reductase inhibitor is lovastatin or lovastatin acid.

37. The method of claim 31, wherein at least about 10 to about 60 mg of the HMG-CoA reductase inhibitor is administered per day.

38. The method of claim 31, wherein said human exhibits symptoms of Alzheimer's Disease.

39. The method of claim 31, further comprising preparing said controlled release formulation by combining said HMG-CoA reductase inhibitor with a pharmaceutically acceptable water swellable polymer and an osmotic agent into a compressed tablet core having at least one coating comprising a pH sensitive agent and a water insoluble polymer.

40. The method of claim 39, wherein said HMG-CoA reductase inhibitor comprises from about 10 to about 60 mg lovastatin or a pharmaceutically acceptable salt thereof.

41. The method of claim 39, wherein said HMG-CoA reductase inhibitor comprises lovastatin acid.

42. The method of claim 39, wherein said formulation further comprises a seal coating applied to the compressed tablet.

43. The method of claim 39, wherein said formulation further comprises a coating layer comprising an enteric coating
